(19) United States
(12) Patent Application Publication
(10) Pub. No.: US 2015/0209511 A1
(43) Pub. Date: Jul. 30, 2015

(54) FLUID TRANSPORT APPARATUS, AND METHOD OF CONTROLLING FLUID TRANSPORT APPARATUS

(71) Applicant: SEIKO EPSON CORPORATION, Tokyo (JP)

(72) Inventor: Yoshihiko MOMOSE, Shiojiri-shi (JP)

(21) Appl. No.: 14/606,952

(22) Filed: Jan. 27, 2015

(30) Foreign Application Priority Data
Jan. 28, 2014 (JP) 2014-013074

(51) Int. Cl. A61M 5/172; A61M 5/142

(52) U.S. Cl. CPC A61M 5/172; A61M 5/142; A61M 2205/3355

(57) ABSTRACT

A fluid transport apparatus includes: a pump configured to change a volumetric capacity of a container in which a fluid is stored to cause the fluid to flow; a flow channel connected to a downstream side of the pump in a direction of flow of the fluid and configured to allow the fluid to flow therein; a pressure transmitting plate arranged so as to be capable of coming into abutment with a first member which constitutes at least part of the flow channel; and a pressure sensor configured to detect a force for displacing the pressure transmitting plate. The fluid transport apparatus is capable of detecting an abnormality in transportation of the fluid.

### BACKGROUND

1. Technical Field

The present invention relates to a fluid transport apparatus configured to transport fluid and a method of controlling the fluid transport apparatus.

2. Related Art

An insulin pump configured to inject insulin into a biological body is now put into practical use. A fluid transport apparatus such as an insulin pump is fixed to the biological body such as human body, and injects a fluid into the biological body such as the human body regularly according to a preset program.

JP-A-2010-48121 discloses a technology for transporting liquid stored in a reservoir by using a liquid transporting unit.

If an abnormality occurs in transportation in the fluid transport apparatus as described above, fluid cannot be transported adequately. Therefore, it is preferable to detect an abnormality in transportation of fluid.

### SUMMARY

An advantage of some aspects of the invention is to detect an abnormality in transportation of a fluid.

An aspect of the invention is directed to a fluid transport apparatus including: a pump configured to change a volumetric capacity of a container in which a fluid is stored to cause the fluid to flow; a flow channel connected to a downstream side of the pump in a direction of flow of the fluid and configured to allow the fluid to flow therein; a pressure transmitting plate arranged so as to be capable of coming into abutment with a first member which constitutes at least part of the flow channel; a pressure sensor configured to detect a force for displacing the pressure transmitting plate.

Other characteristics of the invention will be apparent from the specification and attached drawings.

### BRIEF DESCRIPTION OF DRAWINGS

The invention will be described with reference to the accompanying drawings, wherein like numbers reference like elements.

FIG. 1 is a perspective plan view of a fluid transport apparatus.

FIG. 2 is a perspective side view of the fluid transport apparatus.

FIG. 3 is a first cross-sectional view of a first embodiment taken along a line A-A in FIG. 1.

FIG. 4 is a second cross-sectional view of the first embodiment taken along the line A-A in FIG. 1.

FIG. 5 is a flowchart for explaining control of the fluid transport apparatus.

FIG. 6 is a first cross-sectional view of a second embodiment taken along the line A-A in FIG. 1.

FIG. 7 is a second cross-sectional view of the second embodiment taken along the line A-A in FIG. 1.

### DESCRIPTION OF EXEMPLARY EMBODIMENTS

According to the specification and the attached drawings, at least the followings become apparent. That is, a fluid transport apparatus includes: a pump configured to change a volumetric capacity of a container in which a fluid is stored to cause the fluid to flow; a flow channel connected to a downstream side of the pump in a direction of flow of the fluid and configured to allow the fluid to flow therein; a pressure transmitting plate arranged so as to be capable of coming into abutment with a first member which constitutes at least part of the flow channel; a pressure sensor configured to detect a force for displacing the pressure transmitting plate.

In this configuration, displacement of the first member which constitutes at least part of the flow channel can be detected by the pressure sensor via the pressure transmitting plate, and hence displacement of a tube can be detected further reliably. On the basis of the displacement, an abnormality in transportation of the fluid can be detected.

It is preferable that the fluid transporting apparatus includes a reference value storing unit configured to store a reference value of the force detected by the pressure sensor; and a determining unit configured to determine an occurrence of an abnormality in transportation in the flow channel when the force detected by the pressure sensor is larger than the reference value.

In this configuration, an abnormality in transportation in the flow channel may be determined on the basis of the force detected by the pressure sensor.

It is preferable that the flow of the fluid is stopped when the occurrence of an abnormality in the transportation in the flow channel is determined.

In this configuration, when an abnormality occurs in transportation in the flow channel, the flow of the fluid is stopped, so that an increase in extent of an abnormality in fluid transportation can be restrained.

It is preferable that a window portion having a smaller surface area than that of the pressure transmitting plate is provided, and the pressure sensor detects a force that the first member causes the pressure transmitting plate to be displaced via an opening of the window portion.

In this configuration, an end portion of the pressure transmitting plate may be held at the window portion. Since the surface area of the pressure transmitting plate is large, a larger force is transmitted to the pressure sensor, so that an abnormality of the fluid in transportation may be detected with higher degree of sensitivity.

It is preferable that a second member configured to come into contact with the pressure transmitting plate and transmit a force to a pressure detection unit in the pressure sensor is provided.

It is preferable that the second member has a spherical shape.

It is preferable that the pressure detection unit in the pressure sensor is a semiconductor force sensor element.

It is preferable that the pressure sensor is provided with a spherical shaped member configured to come into contact with the pressure transmitting plate and transmit a force to the semiconductor force sensor element in the pressure sensor.

In this configuration, the spherical shaped member comes into contact with the pressure transmitting plate at one point, and hence the displacement of the pressure transmitting plate can be detected with higher degree of sensitivity.

It is preferable that at least part of the flow channel is a tube.

It is preferable that the tube includes a resiliently deformable member.

In this manner, when the tube is resiliently deformed, the tube is expanded due to clogging, so that the tube can press the pressure sensor via the pressure transmitting plate. Accordingly, the pressure of the tube can be detected with high degree of sensitivity.

It is preferable that the pump includes a syringe and a piston configured to move in the syringe.

In this configuration, fluid may be flowed by changing a volumetric capacity of a container in which the fluid is stored.

It is preferable that at least part of the flow channel includes a groove extending in a direction of flow of the fluid and a film having resiliency, and the first member is the film.

Another fluid transport apparatus including: a pump configured to change a volumetric capacity of a container in which a fluid is stored to cause the fluid to flow; a tube connected to the pump; a pressure sensor arranged on a downstream side of the pump in a direction of flow of the fluid; and a pressure transmitting plate arranged on a side surface side of the tube; wherein the pressure sensor detects a force of the tube to displace the pressure transmitting plate.

In this configuration, displacement of the tube can be detected by the pressure sensor via the pressure transmitting plate, and hence displacement of the tube can be detected further reliably. On the basis of the displacement, an abnormality in transportation of the fluid can be detected.

A method of controlling a fluid transport apparatus includes changing a volumetric capacity of a container in which a fluid is stored to cause the fluid to flow in a flow channel; causing a pressure transmitting plate arranged so as to be capable of coming into abutment with a first member which constitutes at least part of the flow channel to be displaced; and detecting a force for displacing the pressure transmitting plate.

### First Embodiment

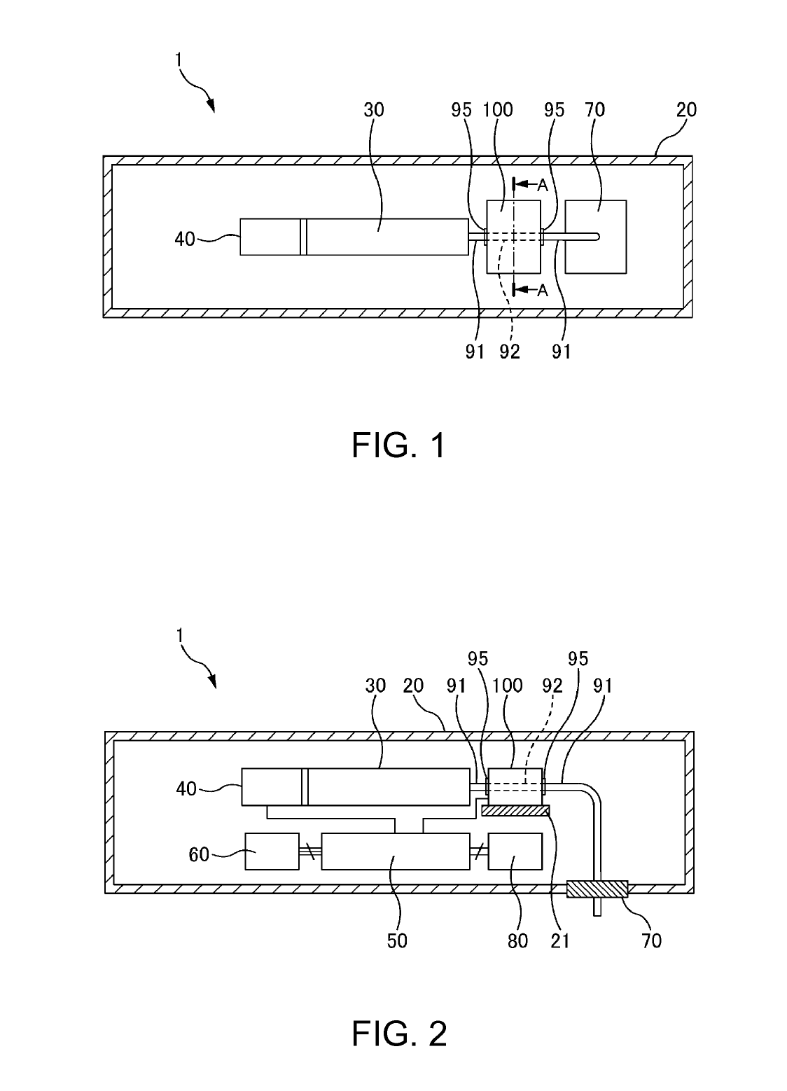

FIG. 1 is a perspective plan view of a fluid transport apparatus 1. FIG. 2 is a cross-sectional side view of the fluid transport apparatus 1. In FIG. 1 and FIG. 2, an interior of the fluid transport apparatus 1 is illustrated in a perspective manner to make respective elements in the interior of the fluid transport apparatus 1 visible.

In FIG. 1 and FIG. 2, a reservoir 30, a dispenser 40, a controller 50, a wireless receiver 60, an outlet port assembly 70, a power source 80, catheters 91 and 92 (which correspond to tubes and may be referred to as cannulas) and a clogging detection unit 100 housed in a housing 20 are shown.

Here, the catheters are denoted by reference numerals 91 and 92, but a catheter denoted by 91 is formed of a material which is relatively difficult to be deformed such as a fluorine resin, and a catheter denoted by 92 is formed of a relatively soft material such as elastomer.

As illustrated in FIG. 1 and FIG. 2, the catheter 92 is disposed within the clogging detection unit 100, while the catheter 91 is disposed at a position other than the interior of the clogging detection unit 100. The catheter 91 and the catheter 92 are connected to each other by a connecting member 95.

Liquid to be administered to the biological body or the like via the catheters 91 and 92 is stored in the reservoir 30. The dispenser 40 is configured to apply a pressure to a drug solution by changing the volumetric capacity in the reservoir 30, and cause the liquid to flow to the catheters 91 and 92. As a method of applying a pressure to the liquid in the reservoir 30, a configuration in which the reservoir 30 is formed into a syringe and a piston is moved in the interior thereof may be employed.

The controller 50 controls respective parts of the dispenser 40 and the fluid transport apparatus 1. The controller 50 includes a memory device in the interior thereof. A reference value, which will be described later, is stored in the memory device. The controller 50 controls the dispenser 40 on the basis of a signal from the clogging detection unit 100 as described later.

The wireless receiver 60 receives an instruction from a remote control apparatus, which is not illustrated. The instruction is sent to the controller 50. The controller 50 controls the dispenser 40 to apply a pressure to the liquid in the reservoir 30 as described above, and feed the liquid to the catheters 91 and 92. Although the wireless receiver is provided here, a configuration in which the fluid transport apparatus 1 is operated independently by the controller 50 instead of wireless remote control is also applicable.

The outlet port assembly 70 holds a portion in the vicinity of an end portion of the catheter 91 adequately. The outlet port assembly 70 also maintains a state in which a distal end of the catheter 91 is inserted stably in the biological body.

The power source 80 supplies power required for the wireless receiver 60 and the controller 50 via the controller 50.

The clogging detection unit 100 sends a pressing force applied by expansion of the catheter 92 to the controller 50. The clogging detection unit 100 is fixed to a predetermined position in the housing 20 by a fixed member 21. Hereinafter, a configuration of the clogging detection unit 100 will be described.

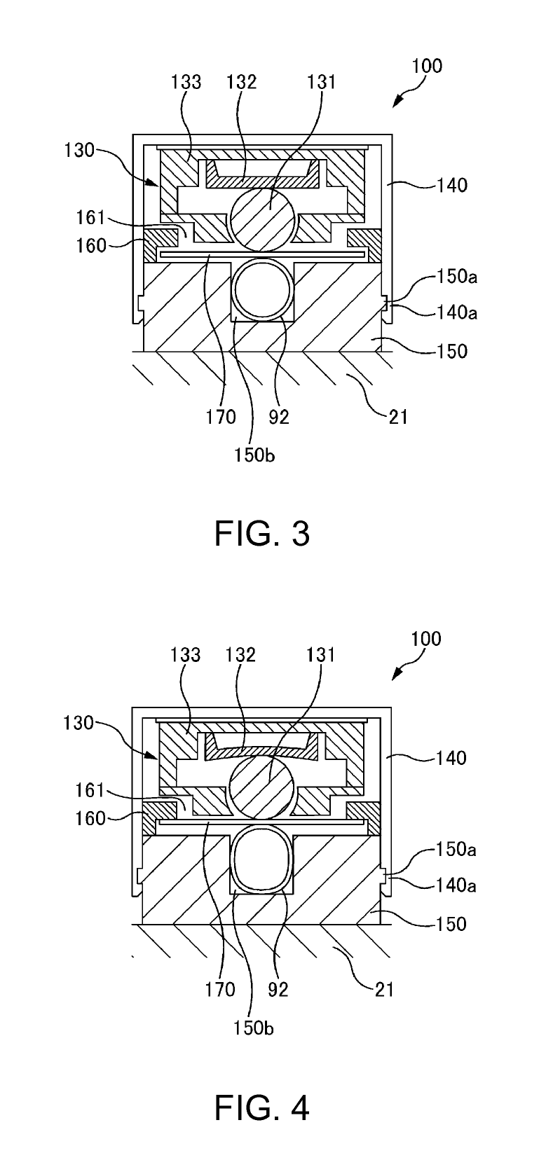

FIG. 3 is a first cross-sectional view taken along a line A-A in FIG. 1. FIG. 4 is a second cross-sectional view taken along the line A-A in FIG. 1. FIG. 3 illustrates a state before the catheter 92 as a flow channel of the liquid is clogged. In contrast, FIG. 4 illustrates a state after the catheter 92 as a flow channel of the liquid has clogged.

The clogging detection unit 100 includes a clogging detection element 130, a lid member 140, a base member 150, a window member 160, and a pressure transmitting plate 170.

The clogging detection element 130 is a pressure sensor. The clogging detection element 130 includes a spherical member 131, a semiconductor force sensor element 132, and a storage member 133 for storing these members. The semiconductor force sensor element 132 is formed of a Si semiconductor substrate configured to detect forces. The semiconductor force sensor element 132 converts an applied force into an electric signal by using a piezoelectric resistance effect and outputs the converted electric signal. The output electric signal is fed to the controller 50. The spherical member 131 is configured to transmit a force to be measured to the semiconductor force sensor element 132.

The base member 150 is a member which serves as a base of the clogging detection unit 100, and is a member to be fixed to a predetermined position in the housing 20 by the fixed member 21. The base member 150 is formed of a groove portion 150b, and the catheter 92 is fitted to the groove portion 150b. Accordingly, the catheter 92 is held on the base member 150 from a lateral direction and from below. When the catheter 92 expands, the displacement caused by the expansion is concentrated to an upward direction.

The window member 160 is secured to the base member 150. The window member 160 includes an opening of a window portion 161 at a center thereof. The pressure transmitting plate 170 is arranged between the window member 160 and the base member 150. A surface area of the pressure transmitting plate 170 is larger than an opening surface area of the window portion 161. Therefore, the pressure transmitting plate 170 is limited in movement between the window portion 161 and the base member 150.

The clogging detection element 130 is fixed to the inner surface of the lid member 140. When the lid member 140 is fixed to the base member 150, one point of the spherical member 131 comes into contact with the pressure transmitting plate 170. The pressure transmitting plate 170 is interposed at the end portion thereof between the base member 150 and the window member 160 so as to allow a slight movement upward and downward.

The pressure transmitting plate 170 comes into contact with the catheter 92 on a surface opposite from a surface with which the spherical member 131 comes into contact. When the lid member 140 is mounted on the base member 150, the catheter 92 and the pressure transmitting plate 170 abuts against each other, and the pressure transmitting plate 170 and the spherical member 131 abuts against each other. When the lid member 140 is attached to the base member 150, a depression 140a of the lid member 140 engages a projecting portion 150a of the base member 150. Accordingly, the position of the lid member 140 with respect to the base member 150 is fixed, and a relative position among the clogging detection element 130, the pressure transmitting plate 170, and the catheter 92 is determined.

In the case where the catheter 91 on the downstream side of the clogging detection unit 100 is clogged and a flow is generated in the catheters 91 and 92 by the dispenser 40, an internal pressure of the catheter 92 is increased. Therefore, the flexible catheter 92 is expanded. When the catheter 92 is expanded, a side surface of the catheter 92 presses the spherical member 131 of the clogging detection element 130 via the pressure transmitting plate 170 of the window portion 161 (FIG. 4). Therefore, the pressure detected by the clogging detection element 130 is monitored by the controller 50, whereby the fact that the catheter 91 on the downstream side of the clogging detection unit 100 is clogged is detected when the pressure is increased to a level higher than a predetermined pressure.

If the pressure transmitting plate 170 is not provided, the catheter 92, which is deformed resiliently, can hardly press the spherical member 131 on which a force concentrates at one point. In contrast, in this embodiment, since the pressure transmitting plate 170 is provided at the window portion 161, the pressing force of the catheter 92 expanded at the window portion 161 is reliably transmitted to the spherical member 131 via the pressure transmitting plate 170. At this time, a force of a magnitude obtained by multiplying the pressure of the catheter 92 by a contact surface area between the catheter 92 and the pressure transmitting plate 170 is transmitted to the spherical member 131. Since the surface area of the pressure transmitting plate 170 is larger than the opening surface area of the window portion 161, a larger contact surface area is secured, and a larger force can be transmitted to the spherical member 131. Therefore, the clogging of fluid such as liquid can be detected with high degree of sensitivity.

In this embodiment, the clogging detection element 130 having the spherical member 131 is used as the pressure sensor. Since the spherical member 131 theoretically comes into contact with the pressure transmitting plate 170 at one point, the clogging detection element 130 can detect the movement of the pressure transmitting plate 170 at a higher degree of sensitivity. Since the spherical member 131 comes into contact with the pressure transmitting plate 170 at one point, the opening surface area of the window portion 161 can be designed to be small.

Since a material of the catheter 92 in the interior of the clogging detection unit 100 is softer than a material of the catheter 91 out of the clogging detection unit 100, if the catheter 91 is clogged on the downstream side of the clogging detection unit 100, the catheter 92 is expanded more than the catheter 91. Accordingly, the clogging of the catheter 91 on the downstream side can be detected with high degree of sensitivity.

An assembly in which a predetermined pressure is generated in the clogging detection element 130 when the lid member 140 and the base member 150 are assembled may also be employed. In this configuration, detection of clogging with higher degree of sensitivity is achieved.

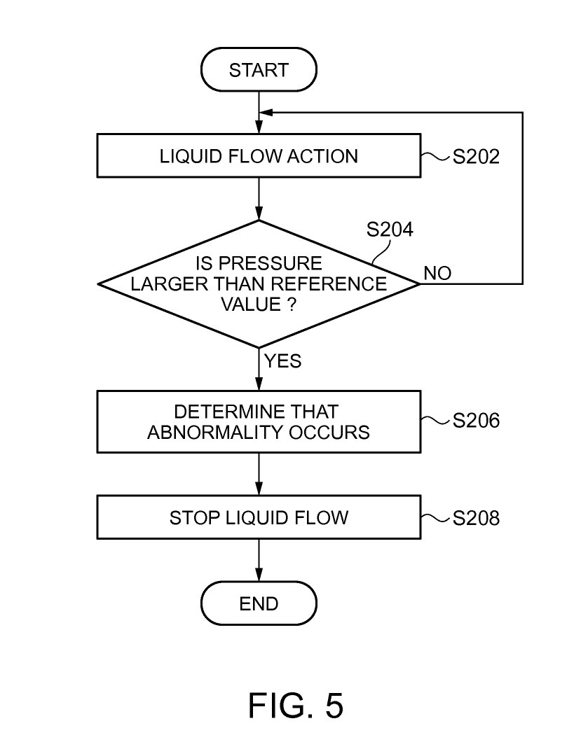

FIG. 5 is a flowchart for explaining control of the fluid transport apparatus 1. An operation of the fluid transport apparatus 1 will be described below with reference to the flowchart.

When the operation of the fluid transport apparatus 1 is started, a flowing action of liquid is started by the dispenser 40 (S202). Accordingly, the liquid in the reservoir 30 is flowed to the catheters 91 and 92.

Subsequently, the controller 50 compares a pressure value sent from the clogging detection element 130 and a reference value stored in advance in the memory device in the controller 50. The reference value is a reference value for determining that an abnormality such as clogging occurs in the catheter 91 on the downstream side of the clogging detection unit 100 when being exceeded. The controller 50 determines whether or not the pressure value exceeds the reference value and, if not, the procedure goes back to Step S202, where the liquid flowing action is continued.

In contrast, when the pressure value exceeds the reference value, the controller 50 determines that an abnormality occurs in transportation of the liquid (S206). The controller 50 sends an instruction to the dispenser 40 for stopping the flow of the liquid. The dispenser 40 stops the flow of the liquid upon reception of this instruction (S208).

In this configuration, since the transportation of the liquid is stopped when an abnormality in transportation of the liquid occurs in the catheter 91 on the downstream side of the clogging detection unit 100, an increase in extent of an abnormality in liquid transportation can be restrained.

### Second Embodiment

In the first embodiment, a configuration in which the spherical member 131 is moved by an expansion of the catheter 92 is employed. However, the spherical member 131 maybe moved via a thin film provided in the flow channel. The configuration in which the spherical member 131 is moved via a film provided in the flow channel will be described.

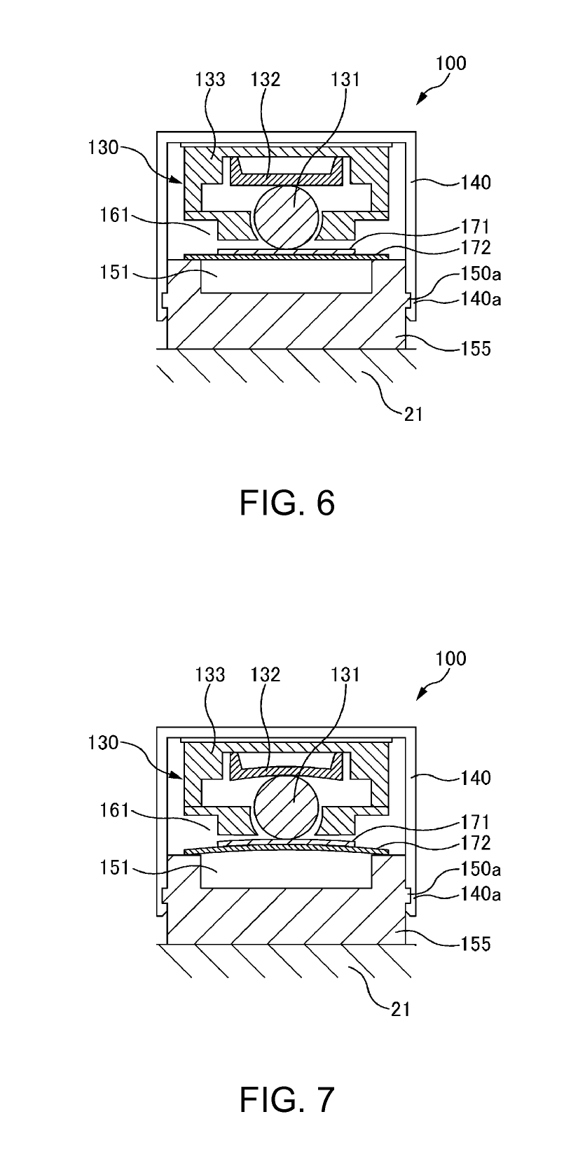

FIG. 6 is a first cross-sectional view of a second embodiment taken along a line A-A in FIG. 1. FIG. 7 is a second cross-sectional view of the second embodiment taken along the line A-A in FIG. 1. In FIG. 6 and FIG. 7, components common to the first embodiment are denoted by the same reference numerals and description will be omitted. A base member in the second embodiment is different in shape from that of the first embodiment, and hence reference numeral 155 is assigned.

In FIG. 6 and FIG. 7, a flow groove 151, a thin plate 171, and a thin film 172 are illustrated in addition to the elements described in the first embodiment. The flow groove 151 is a groove extending in the direction of flow of liquid, and is a groove opened on the upper side of the base member 150. The catheter 91 is connected to both ends of the flow groove 151 by using a connecting member or the like. Accordingly, the liquid sent from the reservoir 30 flows within the flow groove 151.

The thin film 172 is adhered to an upper surface of the base member 150. The thin film 172 is adhered so that an entire circumference except for a position opposing the flow groove 151 comes into contact with the upper surface of the base member 150, whereby the liquid is prevented from leaking from the flow groove 151. In addition, the thin plate 171 is adhered to an upper surface of the thin film 172 at a center thereof. The thin film 172 is a resilient member such as elastomer. The thin plate 171 is formed of a material such as stainless steel.

In this configuration, in the case where the catheter 91 is clogged on the downstream side of the clogging detection unit 100, the thin film 172 is deformed in a direction of pressing the spherical member 131. Since the thin film 172 is provided with the thin plate 171 on an upper surface thereof, the thin plate 171 comes into contact with the spherical member 131, so that upward and downward displacement of the thin film 172 is reliably transmitted. Accordingly, the clogging of the catheter 91 on the downstream side can be reliably detected.

### Other Embodiments

Since the fluid transport apparatus 1 described above may achieve small sizes and thin profiles, and cause a very small amount of flow stably and continuously. Therefore, it is suitable for medical practices such as development of new medicines, or drug deliveries by mounting inside biological bodies or on the surfaces of the biological bodies. The fluid transport apparatus 1 may be used in the several mechanical apparatuses by mounting in the apparatus or in the exterior of the apparatus for transferring fluid such as water, saline solution, drug solution, oils, aromatic liquid, ink, or gas. Furthermore, a micro pump itself may be used for a flow and a supply of fluid as a stand-alone unit.

The spherical member 131 is employed as the member for transmitting forces to the semiconductor force sensor element 132. However, the invention is not limited thereto. Polygonal shapes such as a parallelepiped or a cubic shape are also applicable.

Although the semiconductor force sensor element 132 is used as the pressure sensor in the embodiments described above, the invention is not limited thereto, and any type of pressure sensor is applicable.

In the embodiments described above, the catheters 91 and 92 have been described as being formed of different materials. However, the catheters 91 and 92 may be formed of the same material.

The embodiments described above are for facilitating the understanding of the invention, and are not for interpreting the invention in a limited range. It is needless to say that the invention may be modified or improved without departing the scope of the invention and equivalents are included in the invention.

The entire disclosure of Japanese Patent Application Nos. 2014-13074, filed Jan. 28, 2014 is expressly incorporated by reference herein.

What is claimed is:

1. A fluid transport apparatus comprising:
a pump configured to change a volumetric capacity of a container in which a fluid is stored to cause the fluid to flow;
a flow channel connected to a downstream side of the pump in a direction of flow of the fluid and configured to allow the fluid to flow therein;
a pressure transmitting plate arranged so as to be capable of coming into abutment with a first member which constitutes at least part of the flow channel;
a pressure sensor configured to detect a force for displacing the pressure transmitting plate.

2. The fluid transport apparatus according to claim 1, wherein
the pump includes a syringe and a piston configured to move in the syringe.

3. The fluid transport apparatus according to claim 1, further comprising:
a reference value storing unit configured to store a reference value of the force detected by the pressure sensor; and
a determining unit configured to determine an occurrence of an abnormality in transportation in the flow channel when the force detected by the pressure sensor is larger than the reference value.

4. The fluid transport apparatus according to claim 3, wherein
the flow of the fluid is stopped when the occurrence of the abnormality in the transportation in the flow channel is determined.

5. The fluid transport apparatus according to claim 1, further comprising:
a window portion having a surface area smaller than that of the pressure transmitting plate,
wherein the pressure sensor detects a force that the first member causes the pressure transmitting plate to be displaced via an opening of the window portion.

6. The fluid transport apparatus according to claim 1, further comprising:
a second member configured to come into contact with the pressure transmitting plate and transmit a force to a pressure detection unit in the pressure sensor.

7. The fluid transport apparatus according to claim 6, wherein
the second member has a spherical shape.

8. The fluid transport apparatus according to claim 1, wherein
a pressure detection unit in the pressure sensor is a semiconductor force sensor element.

9. The fluid transport apparatus according to claim 1, wherein
at least part of the flow channel is a tube.

10. The fluid transport apparatus according to claim 9, wherein
the tube includes a resiliently deformable member.

11. The fluid transport apparatus according to claim 1, wherein
at least part of the flow channel includes a groove extending in a direction of flow of the fluid and a film having resiliency, and a first member is the film.

12. The fluid transport apparatus according to claim 1, wherein
the fluid is insulin.

13. A method of controlling a fluid transport apparatus comprising:
changing a volumetric capacity of a container in which a fluid is stored to cause the fluid to flow in a flow channel;
causing a pressure transmitting plate arranged so as to be capable of coming into abutment with a first member which constitutes at least part of the flow channel to be displaced; and
detecting a force for displacing the pressure transmitting plate.
